(19) United States
(12) Patent Application Publication
(10) Pub. No.: US 2007/0243532 A1
(43) Pub. Date: Oct. 18, 2007

(54) System and Method of Detecting a Microorganism

(75) Inventors: Elisabeth Wolf, Erkrath (DE); Wolfgang Breuer, Korschenbroich (DE)

Correspondence Address: COGNIS CORPORATION;PATENT DEPARTMENT, 300 BROOKSIDE AVENUE, AMBLER, PA, 19002, US

(73) Assignee: COGNIS IP MANAGEMENT GMBH, Duesseldorf (DE)

(21) Appl. No.: 10/598,776

(22) PCT Filed: Mar. 3, 2005
(86) PCT No.: PCT/EP05/02209

§ 371 Date: Sep. 11, 2006

(30) Foreign Application Priority Data
Mar. 11, 2004 (DE) 10 2004 011 822.1

(51) Int. Cl. C12Q 1/68

(52) U.S. Cl. 435/6

(57) ABSTRACT

A system for detecting the presence or absence of microorganisms in various types of samples is described. A process for detecting the presence or absence of microorganisms in samples is also described.

### FIELD OF THE INVENTION

This invention relates generally to the detection of microorganisms and the quality control of filterable and/or non-filterable products and to the evaluation of the hygiene status of production plants.

### PRIOR ART

For a long time, the identification of microorganisms in products could only be carried out by time-consuming cultivation and accompanying amplification, the required results only being available after 1 to 2 weeks. For bacteria, fungi and single-cell algae for example, cultivation was carried out in the most favorable nutrient media for each species.

This method of identification establishes how many, and which, microorganisms are present in the end product per unit volume. Living microorganisms capable of proliferating are of particular interest because they can cause unwanted contamination of the intermediate or end product.

A standard method is, for example, membrane filtration where the samples are cultivated and filtered and the microorganisms remain on the membrane. The microorganisms are multiplied and identified on the membrane. Other methods are the standing test and, to an extent, the polymerase chain reaction (PCR). However, since the polymerase chain reaction gives a positive result even with “naked” DNA, false-positive results are commonplace.

However, all these methods are attended by the disadvantage that that the treatment of the samples is very complicated and the result is only known after several days to weeks at the earliest.

Further developments of the known processes have led, for example, to methods which enable living cells in filterable products, such as beverages, to be directly identified by the direct epifluorescent filter technique (DEFT) where fluorescent dyes that bind to DNA and thus block the synthesis of RNA are introduced into the cells and the cells are detected by epifluorescence microscopy (Kroll, R.; Methods in Molecular Biology; 1995; 46; pp. 113-121). EP 386051 and DE 19841588, for example, describe how the DEFT method can be modified by using inductors to form certain enzymes in living microorganisms and subsequently adding a fluorescent reagent which fluoresces by reacting with the enzyme formed and can then be detected. However, this method is only described for the detection of coliform bacteria or lactobacilli. In addition, the DEFT method in any of the known modifications can only be used for filterable products which is a major disadvantage. This method is additionally attended by the problem of the detection limit, at least 10 to 1,000 germs per unit area counted out having to be present. However, such a large number of microorganisms in a sample does not comply with present-day hygiene requirements, so that there is a need for systems which make a smaller number of microorganisms in a sample detectable quickly and without much effort.

For non-filterable products, there is a method which leads to the detection of specific microorganisms by in situ hybridization with fluorescent nucleic acid probes. This method, which is known as fluorescence in situ hybridization (FISH), is used for detecting and locating any kind of nucleic acid in cells. To this end, a molecular hybridization with the DNA/RNA present in chromosomes is carried out using a labeled RNA or DNA probe. (FISH; Amann, R. L., W. Ludwig and K.-H. Schleifer, 1995, Phylogenetic identification and in situ detection of individual microbial cells without cultivation. Microbiol. Rev. 59, pp. 143-169; see also DE 10160666). The specific hybridization of the probe is detected by techniques based on fluorescence microscopy, as described for example in DE 19936875.

The FISH technique is based on the fact that there are certain molecules in bacterial cells which, by virtue of their vital function, were only slightly mutated in the course of evolution: 16S and 23S ribosomal ribonucleic acid (rRNS). Both are constituents of the ribosomes, the sites of protein biosynthesis, and serve as specific markers by virtue of their ubiquitous dispersion, their size and their structural and functional constancy. The rRNA data banks may be used to construct species- and genus-specific gene probes. To this end, all available rRNA sequences are compared with one another and probes which specifically detect a species, genus or group of bacteria are prepared.

In the FISH technique, these gene probes, which are complementary to a certain region on the ribosomal target sequence, are channeled into the cell. The gene probes are generally small, 16-20-base-long single-stranded deoxyribonucleic acid fragments and are directed towards a target region which is typical of a species or group of bacteria. If the fluorescence-labeled gene probe finds its target sequence in a bacterial cell, it binds to that sequence and, by virtue of their fluorescence, the cells can be detected with a fluorescence microscope.

However, studies have shown that, due to large population variations, these methods are attended by statistical problems at the sampling level. The detection limit is again a problem because at least 10 to 1,000 germs per unit area counted out are again necessary for obtaining conclusive results. Such large numbers of microorganisms in a sample do not comply with present-day hygiene requirements.

Although, according to the International Pharmacopoeia (Ph. Eur.), a detection limit of <1000 CFU/g is still acceptable and adequate for the marketing of products, industry and the consumer now expect a far lower detection limit or the hygiene standards products are now expected to meet are so high that a detection limit of <10 CFU/g is inevitable and a detection limit of <1 CFU/g should be targeted.

The methods for detecting and quantifying microorganisms must become increasingly more sensitive and user-friendly to meet the demand for fast and highly efficient detection methods, low detection limits and low cost for many different microorganisms. It is often sufficient merely to perform an “absence test” (yes/no test) as a first step to ascertain whether there are any microorganisms at all in the sample before they are taxonomically determined. It is too time-consuming and requires too much laboratory capacity to have to apply numerous detection methods for the various microorganisms or to have to select the most effective of the various methods available on the market.

Accordingly, the problem addressed by the present invention was to provide a system with which both filterable and non-filterable samples and products could be analyzed and various microorganisms, both living and dead, could be quantitatively detected by a fast method. The system would enable microorganisms to be detected with a detection limit of <10 CFU/g in the selected quantity of sample. The system would not only be specifically applicable to an organism, it would also provide for the general detection of microorganisms in samples and products. The system would also enable the hygiene status of production plants to be monitored.

### DESCRIPTION OF THE INVENTION

The present invention relates to a quality assurance system for the detection of proliferative microorganisms, comprising
a) a system for enriching microorganisms in a sample in an “overnight culture” corresponding to 8 to 24 hours' cultivation under standard conditions according to International pharmacopoeias (for example Ph. Eur.), food laws, cosmetics directives or commercially available indirect methods, such as for example an indirect method where the CO₂ formed during the growth of the microorganisms is determined,
b) a kit for detecting living, damaged or dead microorganisms in filterable and/or non-filterable products containing
   i) at least one reagent containing an inductor and a fluorescent reagent which, with living cells, leads to the formation of a certain enzyme that releases a detectable fluorescent dye by reaction with a specific fluorescent reagent,
   ii) at least one nucleic acid probe for detecting microorganisms by in situ hybridization, the nucleic acid probe being fixed to a fluorescent marker,
      in which a detection limit for proliferative microorganisms of <10 CFU/g is achieved.

In conventional test methods, only 0.1 g and max. 1 g of the sample is generally taken to perform the tests. This gives rise to a serious statistical problem because, ideally, as large a sample volume as possible should be tested. However, conventional test methods only allow samples of the size mentioned to be taken.

Cultivation in “overnight cultures” corresponds on the one hand to the standard methods prescribed in International pharmacopoeias, food laws and cosmetics directives. An overnight culture specifically means that the samples are cultivated for 8 to 24 hours, preferably for 10 to 20 hours and more particularly for 12 to 15 hours. The standard cultivation conditions are set out in the relevant official text. However, minor deviations, for example in the concentrations of the constituents of the nutrient media, the temperature or other parameters of the standard cultivation methods, should be encompassed by the quality assurance system according to the invention. Similarly, any amendments to the official text should be applicable to the system according to the invention. However, the conditions must be documented so that a detection limit for proliferative microorganisms can be determined. Thus, the quantity of sample used is of great importance.

According to the invention, commercially available indirect methods may also be used for enrichment as an alternative standard method to the methods from the International pharmacopoeias, food laws and cosmetics directives. One indirect method, for example, determines the CO₂ formed during the growth of the microorganisms by adsorption to a membrane and photometric determination in the incubation chamber. This method is marketed, for example, under the name of BacT/ALERT® by the BioMérieux company. This method is very sensitive and indicates potential contamination after overnight culture at the latest, so that the analysis result is available after 24 hours at the latest.

In this method, the (optionally diluted) material to be analyzed is introduced under sterile conditions into an ampoule of nutrient medium. The ampoule is placed in a sample cell inside a heatable incubation chamber. The ampoule has a gas-permeable membrane which is connected to a detection chamber. In the detection chamber is an indicator which adsorbs the CO₂ formed during the growth of cells via the membrane and of which the change is photometrically measured in the incubation chamber. The signals obtained are electronically documented for each sample cell and converted into “growth curves” (time vs. CO₂ content).

This method is already being used in medicine for the sterility control of blood bank samples, stored blood, bones, tissue samples and other medically relevant materials.

Its use for the microbiological quality assurance of samples according to the invention has not yet been described or tested in accordance with the invention. The method is suitable for all water-miscible and water-immiscible liquids and for emulsions, wax-containing pearlescent preparations, oils, pastes and solids.

A defined quantity—generally 1 g—of a sample to be analyzed is introduced under sterile conditions into the liquid chamber and incubated at 15 to 40° C. and preferably at 30° C. If the sample contains germs, they will proliferate with evolution of CO₂. For an infestation level of 1 to 100 germs per ml, the evolution of gas can be recorded after about 10 generation cycles and after ca. 2 hours. In positive cases, the material can be immediately further analyzed using the reagents from b i) or b ii) and the corresponding process.

As soon as a positive indication of contamination is present, the type of contamination can be verified.

According to the invention, 1 to 10 g and preferably 5 g of the sample or product to be analyzed are suspended in 100 to 1000 ml standard solution and enriched in the overnight culture. This treatment is often necessary because some samples can have a self-inhibiting effect on microorganisms. In order nevertheless to be able to detect a very low germ population which could destroy the desired quality of the product in relation to hygiene requirements in the event of prolonged storage, there is a need for a method which can still detect the germs after dilution of the sample. However, this involves the problem of the detection limit because the total number of microorganisms for immediate application of the DEFT or FISH test is too small after dilution to be able to carry out an absence test. The first requirement to be satisfied by the quality assurance system in relation to an absence test for proliferative microorganisms or, in other words, a yes/no test for proliferative microorganisms is to ensure that a detection limit of <10 CFU/g is achieved by the overnight culture. More particularly, a detection limit of <1 CFU/g or <1 CFU/5 g is achieved by the sample preparation of 5 to 10 g sample in 100 to 1000 ml standard solution. This is well below the minimum detection limit of <100 CFU/g stipulated by the International pharmacopoeia. The present-day hygiene standard demanded for many products by industry and by the consumer can thus be maintained because a quality assurance system has been developed which enables the smallest populations of proliferative microorganisms to be rapidly detected.

The combination of two detection methods in one kit enables the user to test different samples, intermediate products and end products directly and at the same time. Thus, any intermediate product of whatever consistency can be analyzed for the presence of microorganisms at any phase of a production process.

The detection of microorganisms in accordance with the invention one the one hand involves a “yes/no” test for answering the question of whether unwanted microorganisms are present in the samples or products to be analyzed and, on the other hand, the subsequent exact identification of the detected microorganisms, depending on the sample or product under examination. The particular proof provided depends on the consistency of the sample or product and hence on the reagent to be used and the nucleic acid probe to be used.

In the context of the invention, the terms “samples” and “products” are understood to encompass both intermediate products and end products. In addition, the term “samples” may also be interpreted to include a part of the intermediate product or end product, for example the liquid or solid part of a heterogeneous product or intermediate product, preferably after defined reaction times, more particularly for monitoring an entire production process. In the context of the invention, “samples” are also understood to include, for example, residues of cleaning processes applied to production plants. End products are understood to include both the end product for the consumer and the crude product which is for sale and which is used for the production of end products for the consumer. Samples may also emanate from industrial units for testing effectiveness after disinfection. Industrial units are regularly disinfected with steam or with chemicals (hypochlorites or hydrogen peroxide). Hitherto, effectiveness testing has been carried out, if at all, by standard methods. In many cases, however, effectiveness has been based on empirical values because the standard method is too involved. Using the system and the process according to the invention, the success of disinfection can be tested quickly and effectively.

In the context of the invention, “filterable” sample or product means that the sample or product is able to pass through filters with a pore diameter of 0.45 μm. Accordingly, no oil droplets or solid particles or the like should be present.

In one particular embodiment of the invention, the reagent from b i) of the kit according to the invention is used for filterable liquid samples and products or for filterable liquid components of the samples and products to be analyzed for detecting living microorganisms. Dead microorganisms may also be indirectly detected in this way. This method of detection investigates the metabolic pathway from induction to the formation of an enzyme through the uptake of a specific substance. Induction is performed by a reagent containing an inductor and a fluorescent reagent which is able to pass through the cell membrane, after which the induced enzymes allow the intracellular formation of the highly fluorescent compound. Cells without an intact cell membrane or active metabolism are unable to form the fluorescent reaction product and do not show any fluorescence. By using another colored reagent which accumulates in the dead cells because there can be no reaction through the induced enzyme, it is possible to distinguish between dead and living cells. Only the detection of the enzyme through the formation of the fluorescent reaction product provides the assurance that this metabolic pathway is functioning, so that a functioning metabolism and a proliferative ability can be attributed to those cells. A result is available after only after 1 hour. Membrane filters, more particularly polycarbonate filters with a pore size of 0.2 to 1.20 μm and preferably 0.45 μm, are preferably used to separate the cells from the filterable samples or products. If it is possible, the samples and products to be analyzed may also be suitably liquefied. An inductor of the target enzyme is added to this sample, followed by a fluorescent reagent which only develops its fluorescence after reaction with the induced target enzyme.

These specific fluorescent reagents include, for example, fluorescin digalactoside for detecting galactosidase induced by galactose as inductor. Lactobacilli and coliform bacteria, such as Escherichia coli, Aeromonas, Citrobacter, Enterobacter, Klebsiella, Pseudomonads and other process-water-relevant germs, can be detected with this inductor and fluorescent reagent.

According to the invention, the fluorescent reagents include 4-methyl umbelliferone derivatives specially derivatized for certain enzymes. For example, 4-methyl umbelliferone heptanoate is used for detecting lipase or esterase. 4-Methyl umbelliferone-β-D-galactoside may also be used for detecting galactosidase. The solution is filtered through the described microfilters and fluorescence-optically examined using an epifluorescence microscope.

In another form of application, the indicators and fluorescent reagents are added to the residues on the filter.

In another embodiment, the nucleic acid probe from b ii) of the kit is used both for filterable liquid samples and products and for non-filterable samples and products and for mixtures of filterable and non-filterable samples and products for detecting living microorganisms.

According to the invention, the nucleic acid probe may be a DNA or RNA probe which will generally comprise between 12 and 1000 nucleotides, preferably between 12 and 500, more preferably between 12 and 200, most preferably between 12 and 50 and between 15 and 40 and, in a most particularly preferred embodiment, between 17 and 25 nucleotides. The nucleic acid probes are selected on the basis of whether a complementary sequence is present in the microorganism to be detected. Through the selection of a defined sequence, a species, a genus or a whole group of bacteria can be detected. With a probe of 15 nucleotides, complementarity should exist over 100% of the sequence. With oligonucleotides comprising more than 15 nucleotides, one mispairing site to several mispairing sites are allowed.

The nucleic acid probes from the kit according to the invention are capable of detecting microorganisms non-specifically through non-specific nucleic acid probes. This can clarify the often asked question of whether unwanted microorganisms are present in samples or products without exactly characterizing the microorganism.

Depending on the product and the sample to be analyzed, the hybridization conditions and the hybridization time are adapted in dependence upon the nucleic acid probe.

Suitable detectable markers for the nucleic acid probes are, for example, fluorescent groups such as, for example, CY2 (obtainable from Amersham Life Sciences, Inc., Arlington Heights, USA), CY3 (also obtainable from Amersham Life Sciences), CY5 (also obtainable from Amersham Life Sciences), FITC (Molecular Probes, Inc., Eugene, USA), FLUOS (obtainable from Roche Diagnostics GmbH, Mannheim, Germany), TRITC (obtainable from Molecular Probes, Inc., Eugene, USA), 6-FAM or FLUOS-PRIME.

The quality assurance system according to the invention may be used to detect gram-positive and/or gram-negative bacteria and/or yeast and/or molds and/or algae.

Besides the environmentally relevant species, the gram-positive bacteria also include medically relevant microorganisms, such as for example staphylococci, streptococci, anthrax, tetanus, lactic acid, diphtheria, swine erysipelas and hay bacteria. The gram-negative bacteria also include both environmentally relevant and medically relevant microorganisms, such as gonococci, meningococcil, legionellae, coli, typhus, rur and pest bacteria. The environmentally relevant and human-associated microorganisms include inter alia process-water-specific microorganisms, such as pseudomonads, burkhofderiae, raoultellae, klebsiellae, corynebacteria and bacillus species.

In the microbiological release of end products, the reappearance rate of microorganisms in selectively inoculated products can be determined by the process according to the invention using the reagent from b i) of the kit. Besides bacteria, yeasts and molds can also be detected by applying the process according to the invention. Microorganisms which may be used and detected as standard microorganisms commonly used for selective inoculation—some of which are also named in the International pharmacopoeias—include the following: Pseudomonas aeruginosa, Escherichia coli, Enterobacter cloacae, Staphylococcus aureus, Candida albicans, Aspergillus niger, Salmonella, Bacillus subtilis.

The present invention also relates to the use of the quality assurance system according to the invention for detecting microorganisms and for the quality assessment of filterable and/or non-filterable samples or products and for evaluating the hygiene status of production plants. The filterable and/or non-filterable samples or products to be analyzed are selected from the group consisting of crude products, cosmetic products, pharmaceutical preparations, foods, food supplements, textile auxiliaries, detergents and paints and lacquers.

In the context of the invention, crude products are understood to be products which are used for the manufacture of end products for the consumer. Such products may be surfactants, oil components, emulsifiers, pearlizing waxes, consistency factors, thickeners, superfatting agents, stabilizers, polymers, silicone compounds, fats, waxes, lecithins, phospholipids, UV protection factors, antioxidants, deodorants, antiperspirants, antidandruff agents, film formers, swelling agents, insect repellents, self-tanning preparations, tyrosinase inhibitors (depigmenting agents), hydrotropes, solubilizers, preservatives, perfume oils, non-filterable o/w and w/o emulsions. Process water is also regarded as a crude product.

The cosmetic products may be, for example, ointments, creams, lotions, shampoos, conditioners, shower gels, bath additives, decorative cosmetics, such as make-up, eye shadow, lipstick, nail varnish or the like. The pharmaceutical preparations may be present in the form of juices, creams, ointments, lotions, suspensions, tinctures, drops or the like.

The foods are preferably milk or dairy products, confectionery and meat-based products, beverages, such as mineral water, beer, lemonade or fruit juice. Food supplements are preferably vitamin solutions, unsaturated fatty acids, more particularly conjugated linoleic acids, preservatives or antioxidants.

The present invention also relates to a process for detecting microorganisms in filterable and/or non-filterable products, in which the quality assurance system according to the invention is used by
a) cultivating the samples in an “overnight culture” corresponding to 8 to 24 hours' cultivation under standard conditions according to International pharmacopoeias (for example Ph. Eur.), food laws or cosmetics directives in order to enrich microorganisms,
b) using a kit for detecting living, damaged or dead microorganisms in filterable and/or non-filterable products by
   i) incubating the enriched sample with a reagent containing an inductor and a fluorescent reagent which induces the formation of a certain enzyme in the cells and, in the process, allows a fluorescent compound to be formed from a fluorescent reagent and/or
   ii) after fixing the bacteria, incubating them with a nucleic acid probe which is provided with a fluorescent marker in order to induce hybridization and
c) detecting the fluorescence of the samples and correlating the result with the number of cells, the number of cells being determinable and, where b) i) is used, dead and living cells being distinguishable from one another.

In the context of the invention, “fixing” of the bacteria is understood to be a treatment by which the bacterial envelopes are made permeable to nucleic acid probes. Ethanol is normally used for fixing, although methanol, mixtures of alcohols, a low-percentage paraformaldehyde solution or a dilute formaldehyde solution, enzymatic treatments or the like may also be used.

For “hybridization”, the fixed bacteria are incubated with fluorescence-labeled nucleic acid probes. These nucleic acid probes, which consist of an oligonucleotide and a marker fixed thereto, are then able to penetrate the cell envelope and to bind themselves to the target sequence corresponding to the nucleic acid probe inside the cell. The nucleic acid probes according to the invention may be used with various hybridization solutions. Various organic solvents may be used in concentrations ranging from 0 to 80%. Maintaining stringent hybridization conditions ensures that the nucleic acid probe actually hybridizes with the target sequence. Moderate conditions in the context of the invention are, for example, 0% formamide in a hybridization buffer as described hereinafter. Stringent conditions in the context of the invention are, for example, 20 to 80% formamide in the hybridization buffer.

A typical hybridization solution contains 0 to 80% formamide, preferably 20 to 60% formamide and more particularly 35% formamide. In addition, it has a salt concentration of 0.1 mol/l to 1.5 mol/l, preferably 0.5 mol/l to 1.0 mol/l, more preferably 0.7 mol/l to 0.9 mol/l and most preferably 0.9 mol/l, the salt preferably being sodium chloride. The hybridization solution also typically contains a detergent such as sodium dodecyl sulfate (SDS) for example, in a concentration of 0.001 to 0.2%, preferably in a concentration of 0.005 to 0.05%, more preferably in a concentration of 0.01 to 0.03% and most preferably in a concentration of 0.01%. Various compounds, such as tris-HCl, sodium citrate, PIPES or HEPES, may be used to buffer the hybridization solution, typically in a concentration of 0.01 to 0.1 mol/l and preferably in a concentration of 0.01 to 0.08 mol/l, over a pH range of 6.0 to 9.0 and preferably 7.0 to 8.0. A particularly preferred embodiment of the hybridization solution according to the invention contains 0.02 mol/l tris-HCl, pH 8.0.

The concentration of the probe may vary considerably according to the labeling and number of the expected target structures. In order to achieve rapid and efficient hybridization, the quantity of probe should exceed the number of target structures by several orders of magnitude. However, in the case of fluorescence in situ hybridization (FISH), it is important to bear in mind that an overly large quantity of fluorescence-labeled hybridization probe leads to increased background fluorescence. Accordingly, the quantity of probe should be in the range from 0.5 ng/l to 500 ng/l, preferably in the range from 1.0 ng/l to 100 ng/l and more particularly in the range from 1.0 ng/l to 50 ng/l.

The hybridization time is typically between 10 minutes and 12 hours and preferably ca. 1.5 hours. The hybridization temperature is preferably between 44° C. and 48° C. and more preferably 46° C. The parameter of the hybridization temperature and also the concentration of salts and detergents in the hybridization solution can be optimized in dependence upon the nucleic acid probes, more particularly their lengths and the degree of complementarity to the target sequence in the cell to be detected. After hybridization, the non-hybridized and surplus nucleic acid probe molecules are removed or washed out with a standard washing solution. If desired, this washing solution may contain 0.001 to 0.1% and preferably 0.01 % of a detergent, such as SDS, and tris-HCl in a concentration of 0.001 to 0.1 mol/l, preferably 0.01 to 0.05 mol/l and more particularly 0.02 mol/l. The washing solution also typically contains NaCl in a concentration of 0.003 mol/l to 0.9 mol/l and preferably 0.01 mol/l to 0.9 mol/l, depending on the required stringency. The washing solution may also contain EDTA in a concentration of up to 0.01 mol/l and preferably in a concentration of 0.005 mol/l. In addition, buffer solutions corresponding to the hybridization buffer in a lower salt concentration are added to the washing solution.

The removal of the unfixed nucleic acid probe molecules by washing is normally carried out at a temperature of 30° C. to 50° C., preferably at a temperature of 44° C. to 50° C. and more particularly at a temperature of 46° C. over a period of 10 to 40 minutes and preferably over a period of 15 minutes. Where the kit according to the invention is used, the result is available after 24 to 48 hours. The particular samples or products treated with fluorescent reagent or fluorescent marker are then optically detected using a microscope, preferably an epifluorescent microscope.

### EXAMPLES

### Example 1

### Quality Analysis of Filterable Products using the Described Process Combination The analysis period is generally 10 to 24 hours.

1. Sample Preparation

All work has to be done under sterile/aseptic conditions.

10 g of a sample to be analyzed were weighed in under sterile conditions and homogeneously mixed in 90 ml THLCl bouillon in accordance with Ph. Eur. 2.6.12. or 2.6.13. The solution was sterile-filtered through a 0.45 μl membrane filter and the filtrate was discarded. The filter was rinsed with 200 ml sterile buffer solution. All germs that may be present in 10 g of product were retained on the filter. The filter is then removed and transferred completely to a sterile vessel containing 20 ml CASO bouillon (for enriching bacteria) or Sabouraud bouillon (for enriching yeasts and molds) (standard overnight culture) or to a BacT/ALERT® bottle (lym bottle containing 20 ml of the particular bouillon). The BacT/ALERT® system is not suitable for samples that are known to have a tendency towards the autocatalytic release of CO₂ without the presence of microbial interactions. This is not the case with most cosmetics and industrially used raw materials. The samples thus prepared are incubated overnight at 30±5° C. If there is any suspicion of contamination with slow-growing organisms, the 8-hour pre-enrichment may even be extended to 24 hours.

2. Optical Testing of the Enrichment Culture or Evaluation of the BacT/ALERT System:

If the enrichment bouillon is clouded by microorganism growth or if the evolution of CO₂ can be measured in the BacT/ALERT® system, the sample is further analyzed by the FISH technique and a rough classification into gram-positive and gram-negative organisms is undertaken, possibly even with analysis as far as the germ species (cf. Example 2: non-filterable products). In the absence of growth in the enrichment or in the absence of any CO₂ evolution in the BacT/ALERT® system, the results are verified by the DEFT process.

3. DEFT Test for the Presence of Microorganisms

10 ml of the enrichment culture—corresponding to a quantity of 5 g of starting product—are removed with a sterile syringe and filtered through a 0.45 μm polycarbonate filter. The polycarbonate filter on which the enriched cells lie is placed on a DEFT PAD containing the fluorescent dye and incubated for 8-15 mins. at 20-37° C. in a humid chamber. The dye passes from the PAD onto the cells while the background of the polycarbonate filter is not colored. After incubation, the filter is released from the pad and placed on a slide. The color reaction is then stopped with a fixing liquid. Using a fluorescence microscope with 100× magnification, the preparation is then examined for the presence of colored cells, the filter being completely scanned. The background of the filter must be dark. The positive and negative controls must provide unambiguous results.

4. Result

If red cells are detected, already dead cells were added to the preparation. In general, such cells come from the environment and are not relevant to the quality of a preparation. Products for parental application are excluded. In their case, the preparation should contain neither red nor green fluorescing cells. If green cells are detected, cells capable of proliferating are present in the product. The product has to be classified as microbiologically contaminated. Since the presence of green cells from the preceding enrichment step prevents any conclusions from being drawn as to the germ count in the starting product, the process has to be classified as yes/no or—on the basis of corresponding statistical data—as semi-quantitative. This is generally sufficient for quality assessment because the primary goal is to “detect the absence of proliferative germs”.

3. Testing with Stock Germs by the DEFT Process

carry out sample preparation as described in Point 1 (product sample Texapon NSO, IMQ 418935)
carry negative controls of the enrichment medium used
use 1 series of samples for detecting the stock germs in the presence of the product—addition of 0.2 ml stock germ solution (dilution corresponding to 10-100 CFU/10 ml) to 20 ml sample solution—product or negative control and mix homogeneously
10 ml for standard germ count determination by filtration (HIPCO process) and incubate the filter on CASO medium for 3 to 5 days at 30 to 35° C.
use remaining quantity for enrichment and DEFT, 24 h.
carry out enrichment and evaluation of the test as described in Point 1
no growth-induced clouding or CO₂ formation—DEFT test

3.1 Results—Detection of Stock Germs After Enrichment

| | Germ count determination of | Growth in enrichment and |
|---|---|---|
| | the stock germs used in the | optionally result of the DEFT |
| | filtration process | test after enrichment |
| | CFU/per membrane filter or | CFU per membrane filter or |
| Sample name | per 5 g enriched product | per 5 g enriched product |
| Product sample - enriched | No addition | Enrichment: no clouding |
| | | DEFT: isolated greenish |
| | | crystals, no microorganism cells |
| Negative control sample - | No addition | Enrichment: no clouding |
| medium enriched | | DEFT: isolated greenish |
| | | crystals, no microorganism cells |
| Product sample + E. coli | 50 | Enrichment: serious growth |
| stock solution | | clouding, no DEFT test possible |
| Control sample + E. coli | 51 | Enrichment: serious growth |
| stock solution | | clouding, no DEFT test possible |
| Product sample + Ps. | 46 | Enrichment: no growth clouding |
| aeruginosa stock solution | | DEFT: >1000 green cells |
| Control sample + Ps. | 50 | Enrichment: serious growth |
| aeruginosa stock solution | | clouding, no filtration or |
| | | DEFT test possible |
| Product sample + | 21 | Enrichment: no growth clouding |
| Candida albicans | | DEFT: >1000 (green cells) |
| Control sample + Candida | 23 | Enrichment: no growth clouding |
| albicans | | DEFT: >1000 green cells |

### Example 2

### Quality Analysis of Non-Filterable Products using the Described Process Combination

1. Sample Preparation

All work has to be carried out under sterilelaseptic conditions.

10 g of a sample to be analyzed were weighed in under sterile conditions and homogeneously mixed with suitable aids—preferably a commercially available Stomacher mixer—in accordance with Ph. Eur. 2.6.12. or 2.6.13. in 90 ml THLC1 bouillon. 10 ml of this suspension, corresponding to 1 g of the suspension to be analyzed, were pipetted into 100 ml CASO or Sabouraud bouillon.

It is also possible to transfer 10 ml of the suspension to BacT/ALERT® bottles (lym bottle containing 20 ml of the particular bouillon) which are then placed in the BacT/ALERT® system providing the sample does not show any tendency towards the autocatalytic release of CO₂.

The samples are incubated overnight (max. up to 24 hours) at 30 to 35° C. to allow microbial growth.

2. Optical Testing of the Enrichment Culture or Evaluation of the BacT/ALERT® System:

If the enrichment indicates microbial contamination (bouillon clouding or evolution of CO₂ in the BacT/ALERT® system), the qualitative FISH test is carried out and gram-positive bacteria are distinguished from gram-negative bacteria, which itself can provide an initial indication of the contamination source. The microorganism species are further differentiated either by streak plating on selective media or by PCR processes.

3. Carrying Out the FISH Test for the Presence of Gram-Positive and Gram-Negative Microorganisms (to the Manufacturer's Specification)

Sample Preparation

pipette 100 μl of the sample into an Eppendorf vessel
add 4 drops of solution B2 and mix with sample (=fix)
pipette 10 μl of the fixed samples into the 5 reaction chambers (field 1, 2, 3 and the positive and negative control fields) onto the slide
incubate (dry) slide for 30-60 mins. at 35 to 37° C.
pipette 24 μl breaker solution onto reaction field 3 and incubate for 5-10 mins. at room temperature
add 1 drop breaker 4 to field 3 and I drop breaker 2 to field 2 and incubate for 5-10 mins. at room temperature
fill VIT reactor with demin. water and wash slide
incubate (dry) slide for 30-60 mins. at 35-37° C.
add 1 drop solution B2
incubate slide for 30-60 mins. at 35-37° C.
   Contact with Probes
add 1 drop “positive control” to fields 1, 2 and 3
add 1 drop “negative control” to the negative control field
add 1 drop “positive control G” to the positive control field
insert slide into VIT reactor and incubate for 1.5-2 h at 44-46° C.
   Washing
remove slide from reactor
fill reactor with warm washing buffer, insert slide and wash for 15-30 mins. at 44-46° C.
discard washing buffer
fill reactor with demineralized water and wash slide
dry slide for 15-120 mins. at 44-46° C.
   Microscopic Evaluation
stop reaction with fixing liquid (finisher)
apply glass cover and scan under a microscope with 100× magnification in fluorescent light
background must be dark, positive and negative controls must provide an unambiguous result, luminous red cells are interpreted as living bacteria
   Result:
Field 1=detection of red cells=gram-negative microorganisms (for example genus Pseudomonads, Enterobacteria)
Field 2=detection of red cells=gram-positive microorganisms (for example genus Lactobacilli, Bacillus, Listeria)
Field 3=detection of red cells=gram-positive microorganisms (for example genus Staphylococcus)

If autofluorescences appear in the sample, the procedure has to be repeated and positive control D added. The cells are then green in color, but otherwise no change in the predictions.

Expression of the result calculated per g of product and according to the product-specific limits.

4. Detection of Stock Germs by the FISH Method

carry out sample preparation as in point 1 (product sample: Compound IMQ 322015)
use 1 series of samples for detecting the stock bacteria in the presence of the product—addition of stock bacteria solutions (dilution of the bacteria corresponding to 10-100 CFU/1 g product) to 100 ml CASO bouillon—product sample or negative control—and mix homogeneously

carry out enrichment and test evaluation as in point 2

| | Theoretical germ count | |
|---|---|---|
| | of the stock germs | Result of FISH test |
| | used (calculated) | after enrichment CFU |
| Sample name | CFU per g product | per g enriched product |
| Product sample - enriched | No addition | FISH: fields 1 to 3 no micro- |
| | | organism cells detectable |
| Negative control sample | No addition | FISH: fields 1 to 3 no micro- |
| | | organism cells detectable |
| Product sample + E. coli | 78 | FISH: field 1 - red cells detectable |
| stock solution | | fields 2 to 3 no microorganism |
| | | cells detectable |
| Control sample + E. coli | 78 | FISH: field 1 - red cells detectable |
| stock solution | | fields 2 to 3 no microorganism |
| | | cells detectable |
| Product sample + Ps. | 32 | FISH: field 1 - red cells detectable |
| aeruginosa stock solution | | fields 2 to 3 no microorganism |
| | | cells detectable |
| Control sample + Ps. | 32 | FISH: field 1 - red cells detectable |
| aeruginosa stock solution | | fields 2 to 3 no microorganism |
| | | cells detectable |
| Product sample + | 98 | FISH: field 3 - red cells detectable |
| Staphylococcus aureus | | fields 1 and 2 no micro- |
| | | organism cells detectable |
| Control sample + | 98 | FISH: field 3 - red cells detectable |
| Staphylococcus aureus | | fields 1 and 2 no micro- |
| | | organism cells detectable |

5. Evaluation

Since the presence of cells from the preceding enrichment step prevents any conclusions from being drawn as to the germ count in the starting product, the process has to be classified as “yes/no”. This is generally sufficient for a quality assessment because the primary goal is to “detect the absence of proliferative germs”.

1. A quality assurance system for the detection of proliferative microorganisms, comprising
a) a system for enriching microorganisms in a sample in an “overnight culture” corresponding to 8 to 24 hours' cultivation under standard conditions according to International pharmacopoeias (for example Ph. Eur.), food laws, cosmetics directives or other commercially available indirect methods,
b) a kit for detecting living, damaged or dead microorganisms in filterable and/or non-filterable products containing
i) at least one reagent containing an inductor and a fluorescent reagent which, with living cells, leads to the formation of a certain enzyme that releases a detectable fluorescent dye by reaction with a specific fluorescent reagent,
ii) at least one nucleic acid probe for detecting microorganisms by in situ hybridization, the nucleic acid probe being fixed to a fluorescent marker,
in which a detection limit for proliferative microorganisms of <10 CFU/g is achieved.

2. A quality assurance system as claimed in claim 1, characterized in that a detection limit for proliferative microorganisms of <1 CFU/g is achieved.

3. A kit according to claim 1, characterized in that the reagent from b i) can be used for filterable liquid samples and products or for filterable liquid parts of the samples and products to be analyzed for detecting living microorganisms and indirectly for detecting dead microorganisms.

4. A kit according to claim 1, characterized in that the nucleic acid probe from b ii) can be used both for filterable liquid samples and products and for non-filterable samples and products and for mixtures of filterable and non-filterable samples and products for detecting living microorganisms.

5. A quality assurance system as claimed in any of claims 1 to 4 for detecting gram-positive and/or gram-negative bacteria and/or yeasts and/or molds and/or algae.

6. The use of the quality assurance system claimed in any of claims 1 to 5 for detecting microorganisms and for the quality assessment of filterable and/or non-filterable products and for evaluating the hygiene status of production plants, a detection limit of <10 CFU/g being achieved.

7. The use of the quality assurance system claimed in any of claims 1 to 5 for detecting microorganisms and for the quality assessment of filterable and/or non-filterable products selected from the group consisting of crude products, cosmetic products, pharmaceutical preparations, foods, food supplements, beverages, textile auxiliaries, detergents and dyes and lacquers.

8. A process for detecting microorganisms in filterable and/or non-filterable products, in which the quality assurance system claimed in any of claims 1 to 5 is used by
a) cultivating the samples in an “overnight culture” corresponding to 8 to 24 hours' cultivation under standard conditions according to International pharmacopoeias (for example Ph. Eur.), food laws, cosmetics directives or commercially available indirect methods in order to enrich microorganisms and
b) using a kit for detecting living, damaged or dead microorganisms in filterable and/or non-filterable products by
i) incubating the enriched sample with a reagent containing an inductor and a fluorescent reagent which induces the formation of a certain enzyme in the cells and, in the process, allows a fluorescent compound to be formed from a fluorescent reagent and/or
ii) after fixing the bacteria, incubating them with a nucleic acid probe which is provided with a fluorescent marker in order to induce hybridization and
c) detecting the fluorescence of the samples and correlating the result with the number of cells, the number of cells being determinable and, where b) i) is used, dead and living cells being distinguishable from one another.
